(19) United States
(12) Patent Application Publication
(10) Pub. No.: US 2011/0060320 A1
(43) Pub. Date: Mar. 10, 2011

(54) SYSTEM AND METHOD FOR REPAIRING TISSUE SECTIONS

(76) Inventor: CHEN AHARON-ATTAR, ZIHRON YAAKOV (IL)

(21) Appl. No.: 12/877,065

(22) Filed: Sep. 7, 2010

Related U.S. Application Data
(60) Provisional application No. 61/240,247, filed on Sep. 7, 2009.

(51) Int. Cl. A61B 18/20; A61B 17/03

(52) U.S. Cl. 606/4; 606/213

(57) ABSTRACT

A system and method for repairing a weakness of a tissue, a hole or tear such as in a detachment of a retina in the eye. The system includes a ferromagnetic material which is inserted into the vitreous of an eye of a patient and an external magnet positioned outside the body of a patient arranged to produce magnetic field used for controlling the ferromagnetic material and means for sealing a retinal hole or a retinal tear.

### FIELD OF THE INVENTION

The present invention relates generally to a system and method for repairing weak tissue sections employing ferromagnetic field and more specifically to a system and method for repairing retinal detachment.

### BACKGROUND OF THE INVENTION

Tissue weakness in a vessel, organ and the like is usually a result of a disease, aging, heredity or some other process. Tissue weakness in a vessel may form an aneurysm which is caused due to the pressure of blood flowing though the weakened areas causing the vessel wall to blow up and create an aneurysms sack which tends to enlarge over time and the risk of the sack rupturing increases as the aneurysms grows larger. Acute rupture of the aneurysm is a life-threatening event. Tissue weakness in an organ may be for examples a retinal detachment which is a serious and sight-threatening event, occurring when the retina becomes separated from its underlying supportive tissue. The retina cannot function when these layers are detached, and unless it is reattached soon, permanent vision loss may result.

The retinal detachment is caused by a retinal tear or hole through which liquids from the vitreous pass and detach the retina from the sclera which is the eyeball wall. Typically the causes for retinal detachment are known, yet some detachment are yet to be determined. The know causes are: an injury to the eye or face can cause a detached retina, high levels of nearsightedness, on rare occasions, retinal detachment may occur after LASIK surgery in highly nearsighted individuals, due to cataract surgery, eye-tumors, eye disease, due to systemic diseases such as diabetes and sickle cell disease

The mechanisms for repairing retinal detachment are well known surgical techniques. The mechanisms for repairing retinal detachment are well known surgical techniques. Laser therapy is an ambulatory outpatient procedures. Laser photocoagulation, a method of sealing off leaking blood vessels and destroying new blood vessel growth, maybe useful only at very early stages only. Intraocular gas (i.e. pneumatic retinopexy) or silicon oil are used to tamponade the detachment. Pneumatic retinopexy, in which a bubble of gas is injected into the vitreous humor, the gas bubble expands and is pressed against the retina to hold it against its supportive tissue. The pneumatic retinopexy physically limits the patient to a certain position over a long period of time, which typically lasts between two weeks to two months. If the patient does not follow the head positioning instructions, he or she will risk an aggravation. Moreover, if the detachment area is positioned in the lower parts of the retina or at the close surroundings of the macula, applying pneumatic retinopexy is not possible. Injecting silicone oil or a gas bubble into the eye to keep the detached retina in place requires two surgeries; one for filling the vitreous cavity with the silicon and a second for draining the silicon and filing the vitreous cavity with a suitable gel, these two surgeries are very complicated, require long recovery time and result in poor eyesight during the treatment which may last up to several months. Scleral buckling/ring, in which a silicone band indents the eye to approximate the retina. The tear or hole is closed with supplemental cryotherapy or laser. Cryotherapy (freezing) or photocoagulation will permanently reattach the retina. Scleral buckling/ring surgery is complicated, invasive and require general anesthesia, therefore hospitalizing for several days is inevitable. Scleral buckling/ring surgery requires long recovery time, it may cause post-surgical eye mobility limitations. Intraocular repair with pars plana vitrectomy may be necessary in complicated tractional and exudative detachments, a very complicated, invasive and a long recovery time, with a risk of not recovering the eyesight.

Considering all of the above safety and efficacy requirements, there clearly remains a need for better method and system for repairing a weak section of a tissue or providing tissue support. For examples, in retinal detachment, an advantageous system would repair retinal detachments of different kind and severity without risking the patient's eyesight and providing him easy and short recovery time. An advantageous system will provide the physician with a system the works fast and accurate for saving essential time and money.

### SUMMARY OF THE INVENTION

One embodiment of the present invention is a system for repairing a detachment of a retina in the eye, including a ferromagnetic material which is inserted into the vitreous of an eye of a patient and an external magnet positioned outside the body of a patient arranged to produce magnetic field used for controlling the ferromagnetic material and means for sealing a retinal hole or a retinal tear. The system may also include external heating means such as a laser source positioned outside the body of a patient arranged for heating the fluid confined between the detached retina and the choroid in the eye of a patient resulting in a viscosity change of the confined fluid. The system may also include a microscope for visualizing the ferromagnetic material after it is inserted into the vitreous. The system further includes imaging means for tracking and imaging the ferromagnetic material after it is inserted into the vitreous.

Another embodiment of the present invention is a system for repairing a weakness of a tissue including ferromagnetic material positioned in specific location adjacent to the weak tissue and an external magnet positioned outside the body of a patient arranged to produce a magnetic field used for controlling the ferromagnetic material.

Yet another embodiment of the present invention is a method for repairing retinal detachment including the steps of inserting a ferromagnetic material into a vitreous in the eye of a patient and applying a magnetic field by an external magnet located outside the body for navigating and positioning the ferromagnetic material. After which, applying pressure on the detached retina and sealing a retinal hole or a retinal tear.

### BRIEF DESCRIPTION OF THE DRAWINGS

The invention is herein described, by way of example only, with reference to the accompanying drawings, wherein:

FIGS. 1a,1b,1c,1d,1e,1f and 1g illustrating the principle steps of the procedure for repairing a retinal detachment according to an embodiment of the present invention; and

FIGS. 2a and 2b illustrating several examples of shape and dimensions of a ferromagnetic patch, according to an embodiment of the present invention; and

FIG. 3 illustrating a second embodiment of the present invention, when a retinal detachment is considered to be flat, according to an embodiment of the present invention; and

FIG. 4 illustrating a an example of ferromagnetic patch having at least one pin, according to an embodiment of the present invention.

FIG. 5a illustrating a single pin in an open state and FIG. 5b illustrates the pin in a closed state, according to an embodiment of the present invention.

### DETAILED DESCRIPTION OF THE PREFERRED EMBODIMENTS

The present invention relates to a system and method for repairing weak sections, tear, holes and the like. More specifically for repairing a retinal detachment. The method of the present invention includes inserting a biocompatible ferromagnetic material into a desired section in the human body, navigating and positioning the ferromagnetic material to a specific location and pressing the ferromagnetic material onto the tissue by a magnetic field until the tissue returns to its normal position or until the tissue returns to a required position.

The ferromagnetic material may be for example ferrofluids or a patch, preferably made of biocompatible material having minimal toxicity. Navigating and pressing may be performed by different magnetic field or similar magnetic field, both of which are controlled by an electro-magnet or a magnet, which controls the strength levels of the magnetic field and its orientation. Usually, the magnetic field for pressing is stronger as compared to the magnetic field for navigating.

The desired section of the body to which the ferromagnetic material is inserted must have suitable access to the weakened tissue section. After the ferromagnetic material is inserted it is navigated and positioned in a predetermined location and may also apply pressure on the weak section. The ferromagnetic material may be permanently attached to the tissue. Attaching the ferromagnetic material to the tissue may be carried out by means such as laser, cryosurgery, a second biocompatible ferromagnetic cover, adhesive means which are biocompatible, biocompatible pins and the like.

The ferromagnetic material may be a ferrofluids or a ferromagnetic patch which may cover a hole or a tear, however additional means for sealing a hole or a tear if one exists may also be added. Typically, the ferromagnetic patch shape and dimension concordant the shape and dimension of the weakened tissue area, hole, tears and the like. The ferromagnetic patch may have a permanent shape and dimension or may have a changeable shape and dimension for shrinking and enlarging when required.

Various additional features to the ferromagnetic patch may be added such as various coatings for accelerating or facilitating the biological process of tissue healing such as regeneration. Means for permanently attaching the ferromagnetic patch to a tissue may be biocompatible adhesives such as albumin, bio compatible pins, soldering means such as applying external laser beam and the like. The adhesive substance may be activated by a laser or other activation means such as a illumination of a specific wavelength. The pins also may be activated by ferromagnetic field or by a specific wavelength. The adhesive, pins or the like may be integrated within the patch. If the ferromagnetic patch is temporarily attached it may pulled out of the body of a patient by a syringe. Hence, the temporary patch may be constructed from smaller portions coupled together to a complete patch, and when required, before or during the removal of the patch, it is separated to portions so as to provide easier and safer removal. Alternatively, the patch is capable of being folded and pulled out by a syringe. Additional application may be a patch made of a transparent material so that a laser can be applied through. The ferromagnetic patch may be made from an expandable material, expending after a required period of time.

The system of the present invention for repairing inner tissue tears, holes or weak sections includes an external magnetic field source located outside the body of a patient. A magnetic field source is an electro magnet or a permanent magnet, arranged to produce magnetic field into a body of a patient. The system further includes a ferromagnetic material inserted into the body of a patient, for example into the eye of a patient. The ferromagnetic material is controlled by the magnetic field source. The ferromagnetic material may be attached to the weak sections of the inner tissue, hence strengthening the weak sections and avoiding failure. The ferromagnetic material may be attached permanently or temporarily. A repair of a retinal detachment may also includes means for sealing the retinal hole or tear after the retina returns back to its normal position such means may be an additional ferromagnetic material or a laser procedure for soldering the retina to the choroid. Optional step in the procedure in repairing a retinal detachment involves heating the fluid confined between the retina and the sclera results in a viscosity change of the confined fluid The system may also repair an external tissue.

The present invention may integrate an adhesive substance, such as albumin solution or other tissue adhesive agent to facilitate in attaching a biocompatible patch to a human tissue and more specifically attaching a biocompatible patch to the retina. The adhesive substance may be activated in response to specific wavelength applied on the substance.

The magnetic source for producing magnetic field employed for navigating, positioning and pressuring the ferromagnetic material may be any type of ferromagnetic field generator typically an electro-magnet comprising a ferromagnetic core and coiled wires around it, applying electrical current through the wires will result in a ferromagnetic field with a direction that coincides with the ferromagnetic core and is perpendicular to the wires which dictate the direction of the electrical current.

The principles and operation of a system and method involving a ferromagnetic field for repairing retinal detachment may be better understood with reference to the drawings and the accompanying description.

Before explaining embodiments of the invention in detail, it is to be understood that the invention is not limited in its application to the details of design and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting. Further, where considered appropriate, reference numerals may be repeated among the figures to indicate corresponding or analogous patches.

The term “ferromagnetic material” used hereinafter refers to a single material such as a patch which includes ferromagnetic particles and may also refer to a number of particles such as in ferrofluids.

The term “confined fluid” used hereinafter defined the fluid confined between the detached retina and the choroid.

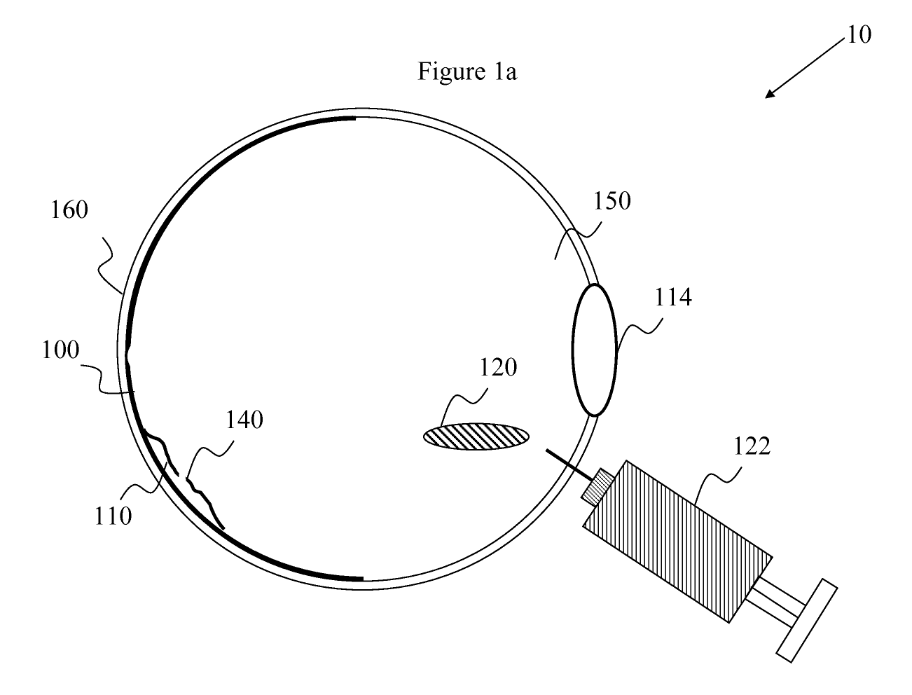

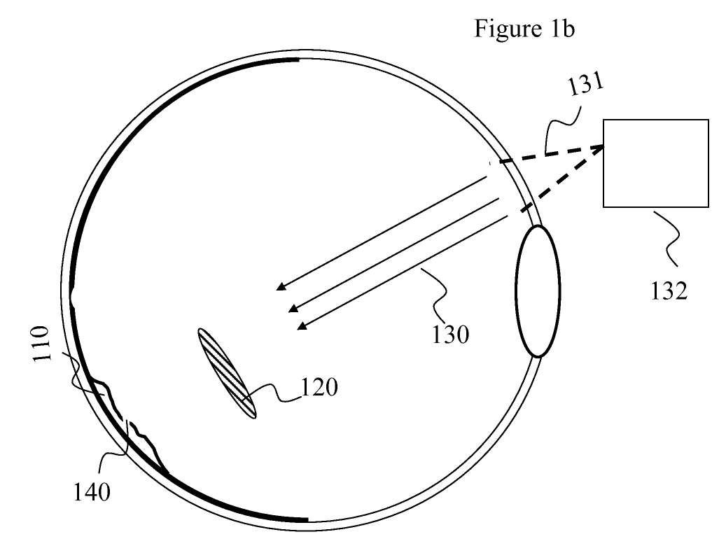

Reference is made to FIG. 1a, FIG. 1b, FIG. 1c, FIG. 1d, FIG. 1e illustrating principle steps of the procedure for repairing a retinal detachment, accordingly FIG. 1a illustrates a section of the an eye of a patient (10) during the procedure of the present invention, including a lens (114), a retina (100) and a choroid (160). The retina (100) is separated from the choroid (160), hence stop receiving essential metabolic support from the retinal pigment epithelium (RPE), resulting in a retinal detachment (110). The separation begins with a hole or a tear in the retina (140) which may result from aging, physical traumatic event, diabetes complication, severe myopia and very occasionally to undetermined reason. The retinal hole or tear provides a pathway for vitreous fluid to pass through and underneath the retina, thus detaching the retina from the choroid. A ferromagnetic material (120) which may be a patch having a required shape, typically the shape and dimensions of the detachment area, is inserted to the vitreous (150) by a syringe (122), yet another option is inserting ferrofluids as the ferromagnetic material.

FIG. 1b illustrates the procedure of navigating the ferromagnetic material (120) towards the retinal detachment location, the navigating procedure is controlled by a magnetic field source (132) producing magnetic field (131). The magnetic field source (132) located outside of the patient body. The magnetic field source (132) may be a permanent magnet or an electromagnet.

FIG. 1c illustrates the procedure of positioning the ferromagnetic material (120) above the retinal hole (140) and the detached retina (110). The positioning procedure is controlled by the magnetic field source (132).

FIG. 1d illustrates the procedure of pressing the detached retina (110) backwards, thus pushing the vitreous fluid confined between the detached retina and the choroid back to the vitreous consequently minimizing or eliminating the detachment area. The pressuring procedure of the ferromagnetic material (120) is controlled by the magnetic field source (132).

FIG. 1e illustrates an example of means for sealing a retinal hole or a retinal tear which after the retina is pushed back to the normal position (110). Such means is a laser source (192) and accompanying optics (193) which introducing a laser beam (191) for soldering the area in the retina which was detached.

FIG. 1f illustrates another example of means for sealing a retinal hole or a retinal tear by an additional ferromagnetic material (194) positioned on the initial ferromagnetic material (120) so as to permanently close the hole as illustrated in FIG. 1g.

FIG. 1h illustrates an example of a patient (170) wearing a magnet (180) which receives power from a power supply unit (182). The magnet is employed for navigating, positioning and pressing the ferromagnetic material which is inserted into the vitreous. The amplitude and orientation of the magnet is controlled by an operator which employs a microscope to visualize the ferromagnetic material inside the eye. The operator may also employ an imaging device for tracking the ferromagnetic material. The patient may be allowed to rest in the hospital or at home during the procedure. The ferromagnetic field strength is adjusted according to the specific case.

The ferromagnetic material is preferably capable of being firmly attached to the retina by means of adhesive or the like, thereby allowing a permanent fixture of the detachment area thereby preventing another detachment.

When the ferromagnetic material is a patch it may be capable of folding and being pulled out of the vitreous by a syringe. The patch may be made from an expandable material, expending after a required period of time (i.e. after eliminating the retinal detachment). The patch may also have an aperture for allowing the confined fluid to pass back to the vitreous. The patch may alternatively not have any apertures or holes. When the ferromagnetic material are ferrofluids it is preferably comprised of ferro-nanoparticle capable of being pulled out by a syringe if necessary or being left inside the eye without interfering. Preferably, the ferrofluids are attached to the retina where the detachment had occurred. The attachment may be carried out by coating the ferrofluids by adhesive substance.

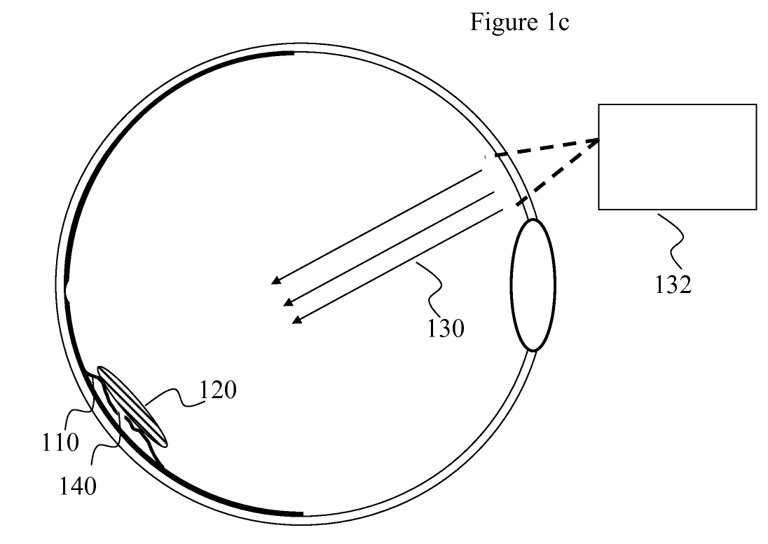

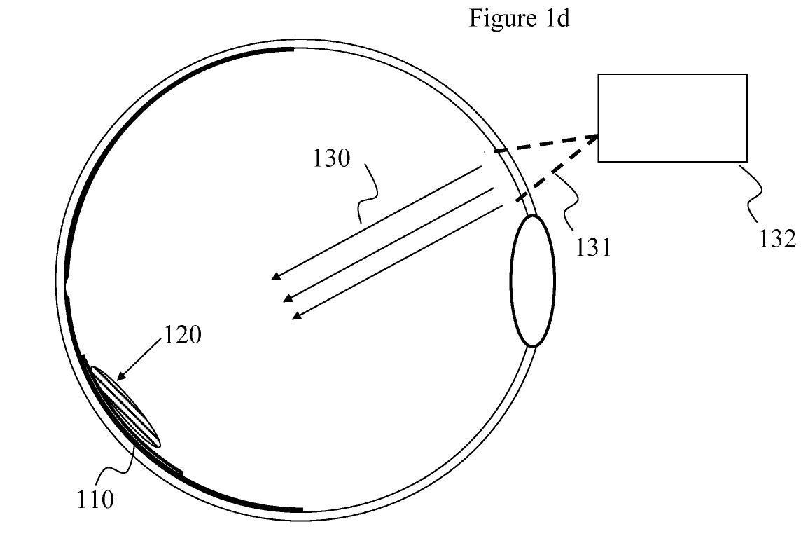

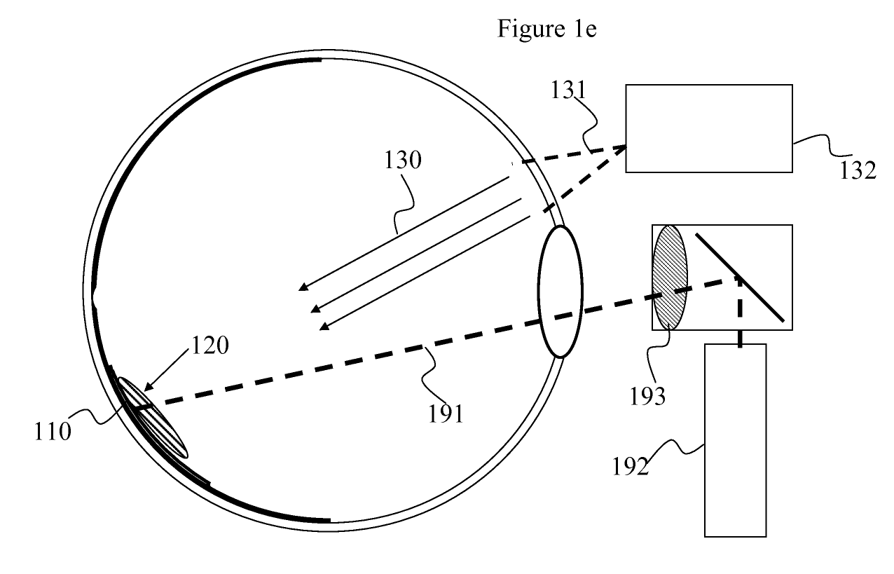

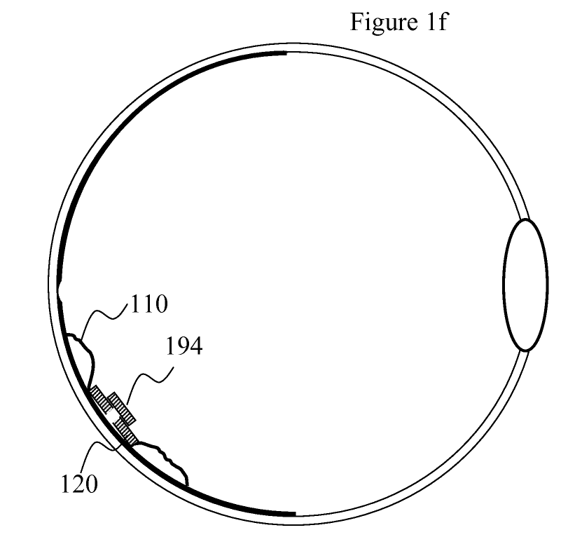

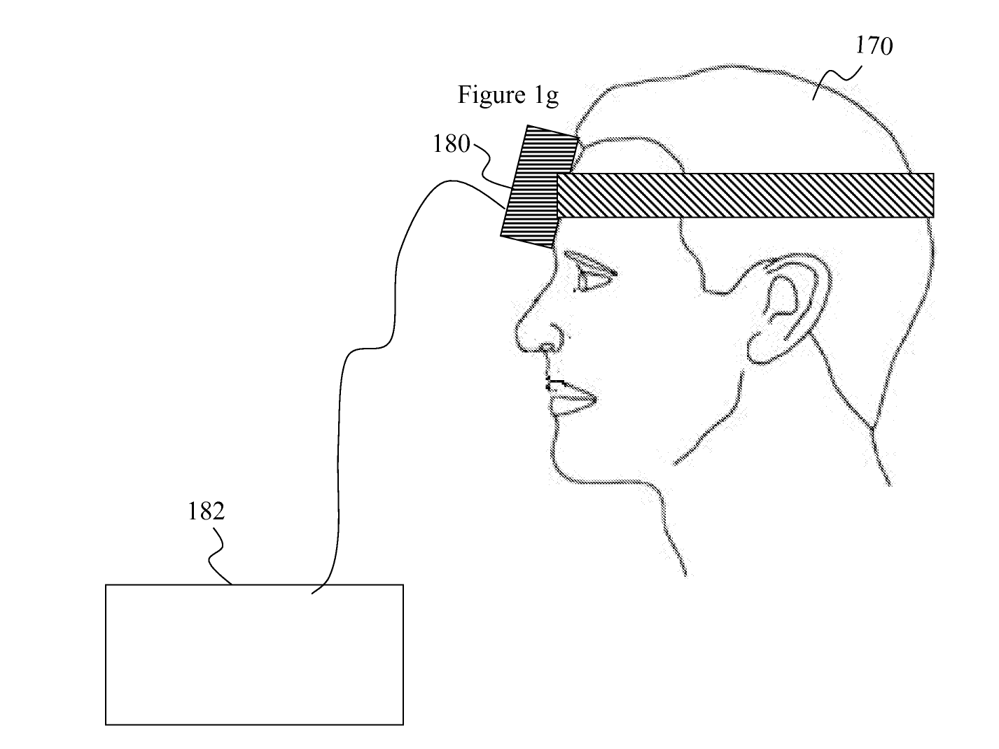

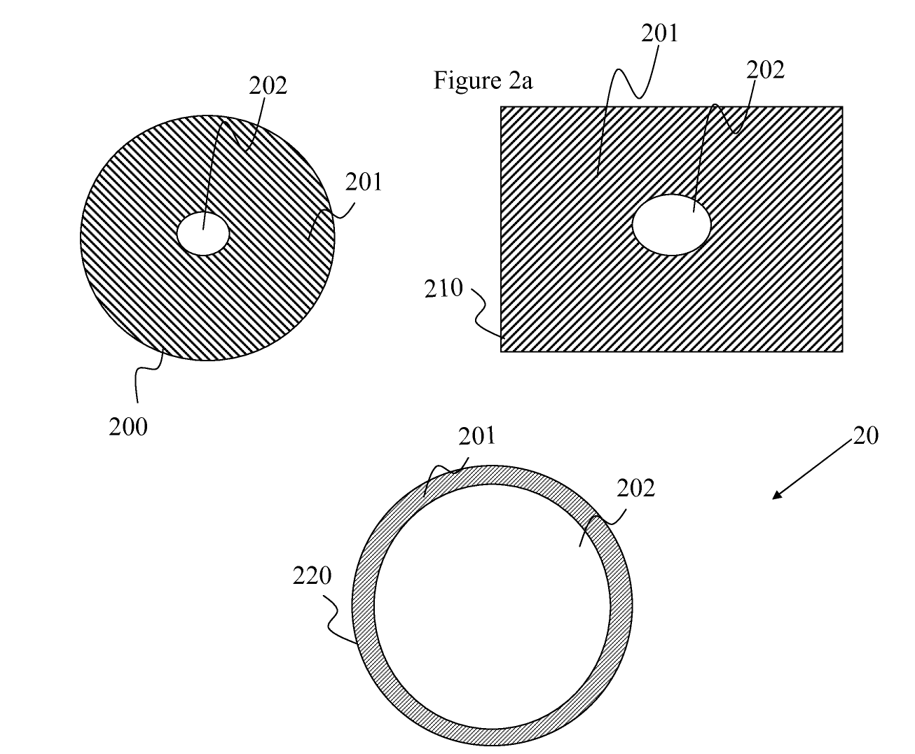

Reference is made to FIG. 2, illustrating several examples of shapes and dimensions of a ferromagnetic patch (20). FIG. 2a illustrates three examples of shapes and dimensions (200,210 and 220) for a ferromagnetic patch, each having ferromagnetic surface (201) and a aperture (202) for providing a pathway for the vitreous fluid to pass through. The shape and dimensions of the patch may vary according to the shape and dimensions of the hole or tear and according to the shape and dimensions of the retinal detachment area. FIG. 2a illustrates the same examples of shapes as previously illustrated (230, 240 and 250), yet having ferromagnetic particles embedded in the surface of the patch. Both types of patches as illustrated in FIG. 2a and FIG. 2b are preferably adhesive or activated to become adhesive. Both types of patches may have a permanent dimension or may have a changeable dimensions for shrinking and enlarging when required.

The patch is preferably made of a transparent biocompatible material. The laser may be applied onto the transparent patch which passes the laser beam onto the retina and allows to simultaneity border the retinal hole and permanently solder the patch to the area where the retina detachment had occurred.

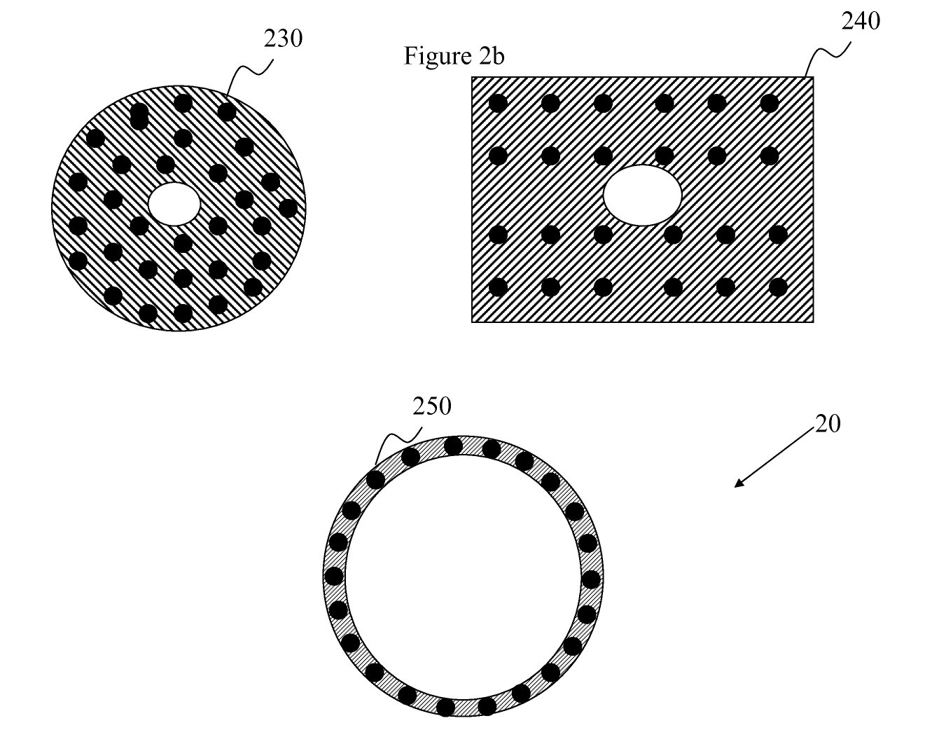

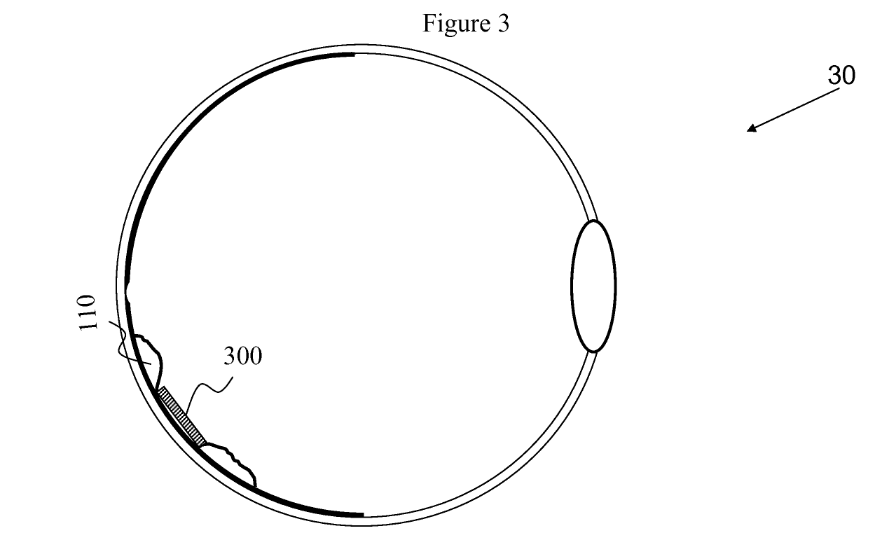

Reference is made to FIG. 3, illustrating a second embodiment of the present invention (30), when retinal detachment is considered to be flat (i.e. relatively low volume of the confined fluid) a whole ferromagnetic patch (300) is employed for slowly pushing the retina backwards to its normal position. The confined fluid diffuses back to the vitreous. The whole patch structure is similar to the ones previously described and differ only in not having any form of an aperture, it is preferably adhesive or activated to become adhesive, further means for attaching may be by laser, cryosurgery and the like.

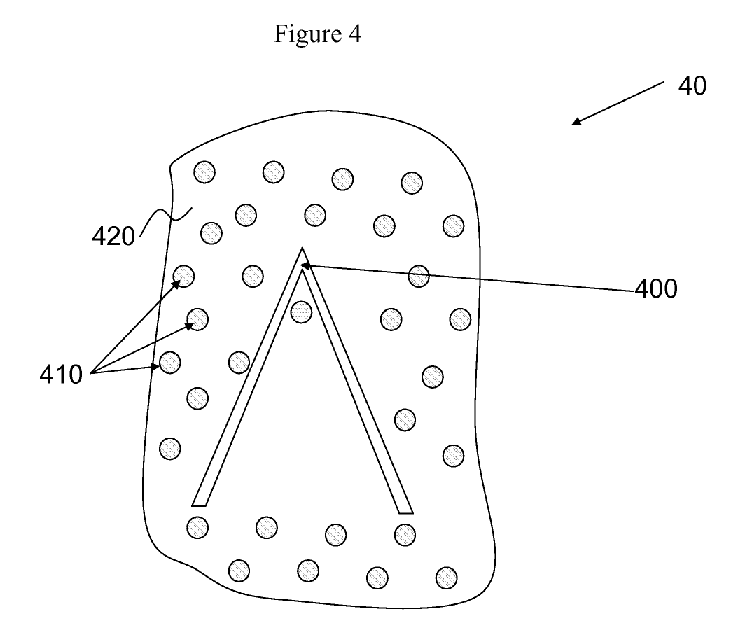

Reference is made to FIG. 4 illustrating a ferromagnetic patch (40) having at least one pin (400) and ferromagnetic magnetic particles (410) embedded in the material of the patch (420). The material may be silicon or other suitable transparent biocompatible substance. Pins are efficient when required to attach a patch to a tissue. Such pins can be integrated to the patch and are almost perpendicular to the patch surface. It is clearly advantageous to use pins that are only activated when needed and before penetration into a tissue for mounting a patch. Pins can be integrated in the patch and may also be part of the patch by applying small cuts in various shapes (V shape for example).

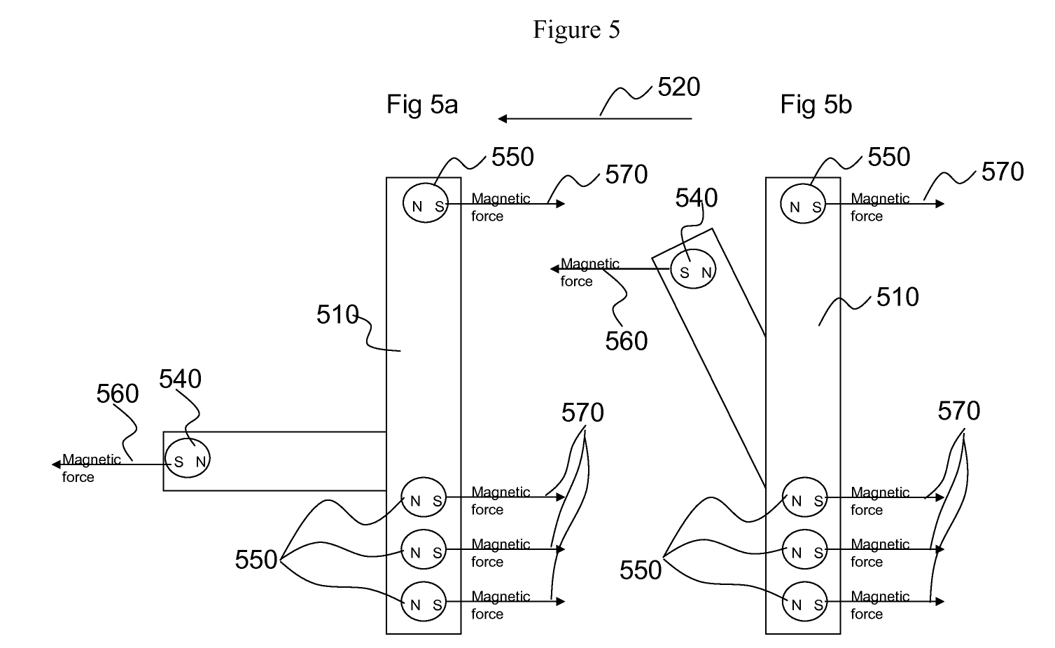

Reference is made to FIG. 5a illustrating a patch having at least one pin (510) in a closed state and FIG. 5b illustrating a patch having at least one pin (510) in an open state. The pin can be folded out or back in the plane of the patch according to the magnetic field direction (520). When embedding miniature ferromagnetic particles within the patch (530), it is possible to set the ferromagnetic particles with different polarities in the main areas of the patch and in the tips of the pins. For example, the polarity of the particle embedded in the pin (540) is opposite to the polarity of the particles embedded in the main patch (550). Using such a distribution will cause forces on the particles (540 and 550) in opposite directions respectively (560 and 570), this results in moving the pin to an open or closed state when applying an external magnetic field.

What is claimed is:

1. A system for repairing a detachment of a retina in the eye, the system comprising:
a ferromagnetic material inserted into the vitreous of an eye of a patient; and
an external magnet positioned outside the body of a patient arranged to produce magnetic field used for controlling the ferromagnetic material; and
means for sealing a retinal hole or a retinal tear.

2. The system according to claim 1, further comprises external heating means positioned outside the body of a patient arranged to heat the fluid confined between the detached retina and the choroid in the eye of a patient, resulting in a viscosity change of the confined fluid.

3. The system according to claim 1, further comprises a microscope for visualizing said ferromagnetic material after it is inserted into the vitreous.

4. The system according to claim 1, further comprises imaging means for tracking and imaging said ferromagnetic material after said ferromagnetic material is inserted into the vitreous.

5. The system according to claim 1, wherein said magnetic field navigates and positions said ferromagnet material in a predetermined location inside the eye.

6. The system according to claim 1, wherein said external magnet arranged to produce said magnetic field which forces said ferromagnetic material in applying pressure on the detached retina.

7. The system according to claim 1, wherein said means for sealing is performed by an additional ferromagnetic material designed for said sealing.

8. The system according to claim 1, wherein said means for sealing is preformed by applying a laser beam around said retinal hole, for soldering the retina surrounding said hole.

9. The system according to claim 1, wherein said ferromagnetic material comprises means for firmly attaching said ferromagnetic material to the retina, thereby allowing a permanent fixture of said detachment.

10. The system according to claim 9, wherein said means for permanently attaching said ferromagnetic material is an adhesive coating.

11. The system according to claim 9, wherein said means for permanently attaching said ferromagnetic material are pins integrated within said ferromagnetic material.

12. The system according to claim 1, wherein said ferromagnetic material is coated with a substance which accelerates the biological process of repairing a tissue.

13. The system according to claim 1, wherein said ferromagnetic material is ferromagnetic particles embedded in a transparent matrix.

14. The system according to claims 11 and 13, wherein said pins comprise magnetic particles having different magnetic polarity orientation relative to the magnetic particles which are integrated to other sections of said matrix.

15. The system according to claim 13, wherein said matrix has an aperture for allowing a passage of the fluid confined between the detached retina and the choroid, through said retinal hole back to said vitreous of the eye.

16. The system according to claim 1, wherein said ferromagnetic material are ferrofluids.

17. A system for repairing a weakness of a tissue, the system comprising:
a ferromagnetic material positioned in specific location adjacent to the weak tissue; and
an external magnet positioned outside the body of a patient arranged to produce a magnetic field used for controlling the ferromagnetic material.

18. The system according to claim 17, further comprises means for attaching said ferromagnetic material to the weak tissue.

19. The system according to claim 17, wherein said ferromagnetic controlling includes navigating, positioning said ferromagnetic material in a required position and for pressuring said ferromagnetic material against said weak tissue area.

20. The system according to claim 19, wherein said means for permanently attaching said ferromagnetic material is an adhesive coating.

21. The system according to claim 19, wherein said means for permanently attaching said ferromagnetic material are pins integrated within a matrix having ferromagnetic particles.

22. The system according to claim 17, wherein said ferromagnetic material are ferrofluids.

23. A method for repairing retinal detachment, comprising the steps of:
Inserting a ferromagnetic material into a vitreous in the eye of a patient; and
applying a magnetic field on the ferromagnetic material by an external magnet located outside the body for navigating and positioning the ferromagnetic material.
applying pressure on the detached retina by said at least one ferromagnetic material operated by a magnetic field.
sealing a retinal hole or a retinal tear.
